SEQUENCE LISTING (2 sequences)

SEQ ID NO: 1 - DNA, 23 bp, Artificial Sequence
  PCR primer for amplification of mitochondrial cytochrome c oxidase subunit 1 DNA region common to rat and mouse
cacaaagatatcggaaccctcta

SEQ ID NO: 2 - DNA, 25 bp, Artificial Sequence
  PCR primer for amplification of mitochondrial cytochrome c oxidase subunit 1 DNA region common to rat and mouse
aagtgggcttttgctcatgtgtcat

(19) United States
(12) Patent Application Publication
(10) Pub. No.: US 2002/0106354 A1
(43) Pub. Date: Aug. 8, 2002

(54) CELLULAR AND ANIMAL MODELS FOR DISEASES ASSOCIATED WITH ALTERED MITOCHONDRIAL FUNCTION

(76) Inventors: CHRISTEN M. ANDERSON, ENCINITAS, CA (US); WILLIAM CLEVENGER, OCEANSIDE, CA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 09/301,517

(22) Filed: Apr. 28, 1999

Related U.S. Application Data
(63) Continuation-in-part of application No. 09/069,489, filed on Apr. 28, 1998.

(51) Int. Cl. A61K 48/00; C12P 21/04; C12N 5/08

(52) U.S. Cl. 424/93.21; 424/93.3; 435/69.1; 435/70.2; 435/320.1; 435/325; 435/366; 435/440

(57) ABSTRACT

The present invention provides methods for depleting mitochondrial DNA from insulin secreting cells using antiviral compounds, and for producing mitochondrial cytoplasmic hybrid (“cybrid”) cells and animals from mitochondrial DNA depleted cells. Also provided are biological models for diseases associated with altered mitochondrial function, including NIDDM, and methods for diagnosis of such diseases and methods for screening agents useful for treating such diseases. Also provided are biological models and methods for evaluating an antiviral compound for its suitability for use in treating a virally-infected patient having a disease associated with impaired insulin secretion, and for evaluating modifications to antiviral compounds in order to determine if such modifications alter (e.g., ameriolate or exacerbate) undesirable side-effects associated with the antiviral compound.

### CROSS-REFERENCE TO RELATED APPLICATIONS

This application is a Continuation-In-Part of U.S. application Ser. No. 09/069,489, filed Apr. 28, 1998.

### TECHNICAL FIELD

The present invention relates generally to model systems for diseases that involve defects in the function of mitochondria, where those defects arise from defects in the genes that regulate mitochondrial structure and activity.

### BACKGROUND OF THE INVENTION

A number of degenerative diseases are thought to be caused by or to be associated with alterations in mitochondrial metabolism. These include diabetes mellitus, Alzheimer's Disease, Parkinson's Disease, Huntington's disease, dystonia, Leber's hereditary optic neuropathy (LHON), schizophrenia, and myodegenerative disorders such as “mitochondrial encephalopathy, lactic acidosis, and stroke” (MELAS), and “myoclonic epilepsy ragged red fiber syndrome” (MERRF), NARP (Neuropathy; Ataxia; Retinitis Pigmentosa), MNGIE (Myopathy and external ophthalmoplegia; Neuropathy; Gastro-Intestinal; Encephalopathy), Kearns-Sayre disease, Pearson's Syndrome, PEO (Progressive External Ophthalmoplegia); congenital muscular dystrophy with mitochondrial structural abnormalities, Wolfram syndrome (DIDMOAD, Diabetes Insipidus, Diabetes Mellitus, Optic Atrophy, Deafness), Leigh's Syndrome, fatal infantile myopathy with severe mitochondrial DNA (mtDNA) depletion, benign “later-onset” myopathy with moderate reduction in mtDNA, dystonia, arthritis, and mitochondrial diabetes and deafness (MIDD).

Type II diabetes mellitus is a common degenerative disease affecting 5 to 10 percent of the population in developed countries. It is a heterogenous disorder with a strong genetic component; monozygotic twins are highly concordant and there is a high incidence of the disease among first degree relatives of affected individuals. The propensity for developing type II diabetes mellitus is reportedly maternally inherited, suggesting a mitochondrial genetic involvement. (Alcolado, J. C. and Alcolado, R., Br. Med. J. 302:1178-1180 (1991); Reny, S. L., International J. Epidem. 23:886-890 (1994)).

Studies have shown that diabetes mellitus may be preceded by or associated with certain related disorders. For example, it is estimated that forty million individuals in the U.S. suffer from late onset impaired glucose tolerance (IGT). Individuals with IGT fail to secrete insulin normally in response to a glucose challenge. A small percentage of IGT individuals (5-10%) progress to non-insulin dependent diabetes (NIDDM) each year. Some of these individuals eventually require therapy with insulin. This form of diabetes is associated with impaired release of insulin by pancreatic beta cells and/or a decreased end-organ response to insulin. Complications of diabetes mellitus and conditions that precede or are associated with diabetes mellitus include: obesity, vascular pathologies, peripheral and sensory neuropathies, blindness, and deafness.

Due to the strong genetic component of diabetes mellitus, the nuclear genome has been the main focus of the search for causative genetic mutations. However, despite intense effort, nuclear genes that segregate with diabetes mellitus are rare and include, for example, mutations in the insulin gene, the insulin receptor gene, the adenosine deaminase gene and the glucokinase gene. A growing body of evidence suggests that the genetic basis of NIDDM resides in mitochondrial DNA rather than in the nucleus. For example, the murine mitochondrial genome, and/or one or more RNAs or proteins encoded thereby, has been shown to be required for the normal regulation of glucose-stimulated insulin secretion in the mouse pancreatic beta cell line MIN6 (Soejima et al., J. Biol. Chem. 271:26194-26199, 1996).

In humans, NIDDM exhibits a predominantly maternal pattern of inheritance and is also present in diseases known to be based on a mitochondrial DNA (mtDNA) defect. Approximately 1.5% of all diabetic individuals, for instance, harbor a mutation at mtDNA position 3243 in the mitochondrial gene encoding leucyl-tRNA (tRNA^{Leu}). This mutation is known as the MELAS (mitochondrial encephalopathy, lactic acidosis and stroke) mutation. (Gerbitz et al., Biochim. Biophys. Acta 1271:253-260, 1995.) Similar theories have been advanced for analogous relationships between mtDNA mutations and other neurological diseases, including but not limited to Leber's hereditary optic neuropathy (LHON), schizophrenia, and myoclonic epilepsy ragged red fiber syndrome (MERRF). It is plausible that other mtDNA mutations are associated with the common form of NIDDM. Identification of such mutations and their functional consequences may provide targets for development of therapeutic agents.

Functional mitochondria contain gene products encoded by mitochondrial genes situated in mtDNA and by extramitochondrial genes such as those found in nuclear DNA. Accordingly, mitochondrial and extramitochondrial genes may interact directly, or indirectly via gene products and their downstream intermediates including but not limited to metabolites, catabolites, substrates, precursors, cofactors and the like. Alterations in mitochondrial function, for example impaired electron transport activity, defective oxidative phosphorylation or increased free radical production, may therefore arise as the result of defective mtDNA, defective extramitochondrial DNA, defective mitochondrial or extramitochondrial gene products, defective downstream intermediates or a combination of these and other factors.

Regardless of whether a defect underlying altered mitochondrial function may have mitochondrial or extramitochondrial origins, and regardless of whether a defect underlying altered mitochondrial function has been identified, the present invention provides methods that are useful for modeling diseases associated with such altered mitochondrial function.

The identification of therapeutic regimens or drugs that are useful in the treatment of disorders associated with altered or defective mitochondrial function such as those described above has historically been hampered by the lack of reliable model systems that could be used for rapid and informative screening of candidate compositions. Animal models do not exist for many of the human diseases that are associated with altered or defective mitochondrial function or mitochondrial gene defects. In addition, appropriate cell culture model systems are either not available, or are very difficult to establish and maintain. Furthermore, even when cell culture models are available, it is often not possible to discern whether the mitochondrial or the cellular genome is responsible for a given phenotype, because mitochondrial functions may often be encoded by both genomic and mitochondrial genes as described above. It is therefore also not possible to tell whether the apparent effect of a given drug or treatment operates at the level of the mitochondrial genome or elsewhere.

In order to determine whether a mitochondrial gene defect may contribute to a particular disease state, it may be useful to construct a model system in which the nuclear genetic background may be held as a constant while the mitochondrial genome is modified. It is known in the art to essentially completely deplete mitochondrial DNA from cultured cells to produce ρ⁰ cells, thereby preventing expression and replication of mitochondrial genes and inactivating mitochondrial function. See, for example, International Publication Number WO 95/26973, which is hereby incorporated by reference in its entirety, and references cited therein. It is further known in the art to repopulate such ρ⁰ cells with mitochondria derived from foreign cells in order to assess the contribution of the donor mitochondrial genotype to the respiratory phenotype of the recipient cells. Such cytoplasmic hybrid cells, containing genomic and mitochondrial DNAs of differing biological origins, are known as cybrids. Additionally, for the production of cybrid cell lines it is known to generate ρ⁰ cells from undifferentiated, immortalized cell lines that can be induced to differentiate in vitro. Generation of cybrid animals by production of ρ⁰ embryonal cells that may be reintroduced into a surrogate mother for completion of gestation, is also known in the art.

Mitochondrial transformations of ρ⁰ cells to produce cybrids known in the art may not always have been done using cells of the types that are most affected by the particular mitochondria associated disease under investigation, making it unclear whether the mitochondrial deficiencies observed in the cybrid cells are related to the disease state being studied.

Clearly, there is a need for reliable model biological systems that may be useful for screening candidate therapeutic compositions and identifying those that may be suitable for treatment of mitochondria associated diseases, including but not limited to diabetes mellitus and neurodegenerative disorders. Such model systems may include in vitro models for these mitochondria associated diseases (e.g., a NIDDM cell line that exhibits impaired insulin secretion or decreased insulin responsiveness); they may also include animal models of these disorders (e.g., an animal model of diabetes mellitus). Reliable diagnoses of mitochondria associated diseases at their earliest stages are critical for efficient and effective intercession and treatment of these disorders, given their often debilitating nature. Accordingly, there is also a need for a non-invasive diagnostic assay that is reliable at or before the earliest manifestations of symptoms for any of the mitochondria associated diseases.

Treating mammals, including humans, with antiviral compounds often has undesirable side-effects. Some of these side-effects appear to occur more frequently or have a greater impact in patients having a disease associated with impaired insulin secretion. For example, treatment with ddC (zalcitabine) can result in a toxic and painful neuropathy, particularly among diabetics (Blum et al., Neurology 46:999-1003, 1996). Moreover, such side-effects can include symptoms resembling those found in patients having a disease associated with impaired insulin secretion. Treatment with ddI, for example, can result in symptoms and conditions resembling diabetes mellitus (Moyle et al., Quarterly Journal of Medicine 86:155-163, 1993; Vittecq et al., AIDS 8:1351, 1994; Munshi et al., Diabetes Care 17:316-317, 1994). Treatment with ddI can result in pancreatitis and pancreatic dysfunction (Seidlin et al., AIDS 6:831-835, 1992). Treatment with AZT can result in myopathy (Garcia et al., J. Clin. Invest. 102:4-9, 1998). Stavudine (d4T), ddI and ddC can cause axonal peripheral neuropathy (Faulds et al., Drugs 44:94-116, 1992; Whittington et al., Drugs 44:656-683, 1992; Browne et al., J. Infect. Dis. 167:21-29, 1993). Impairment of mitochondrial DNA replication has been implicated in the generation of many of such adverse side-effects (Chen et al., Mol. Pharmacol. 36:625-628, 1991; Lewis and Dalakas, Nature Med. 1:417-422, 1995). A major problem with some of these side-effects is their time dependency and therefore delayed onset. Although reversal of toxic side-effects is sometimes seen after cessation of treatment with the antiviral compound(s), in other cases, toxicity has persisted despite the discontinuation of treatment, sometimes with fatal outcomes (Brinkman et al., AIDS 12:1735-1744, 1998).

Clearly, there is a need for model biological systems that can be used to characterize the molecular basis of these and other undesirable side-effects of antiviral treatment and to develop agents that ameriolate such side-effects. Such model biological systems may also be used to screen for and develop drugs that are chemically modified derivatives of antiviral agents that do not cause such side-effects but retain their antiviral activity.

The present invention satisfies these needs for in vitro and in vivo model biological systems that are useful for the development of drug screening assays, diagnostic assays and effective treatment of mitochondria associated diseases, and provides related advantages as well.

### SUMMARY OF THE INVENTION

According to the present invention, model systems for diseases that involve altered mitochondrial function are provided. In one aspect, the invention provides a method of generating a ρ⁰ cell by contacting an insulin secreting cell with an antiviral compound. In another aspect, the invention provides a method of generating a mitochondrial DNA depleted cell by contacting an insulin secreting cell with an antiviral compound. In certain embodiments of these aspects of the invention, the antiviral compound is a nucleoside, nucleotide or base analog or a prodrug thereof, which may in some further embodiments 2′,3′-dideoxycytidine (ddC , 3′-azido-3′ deoxythymidine (AZT, e.g., zidovudine or ZDV), 2′,3′-dideoxyadenosine (ddA), 2′,3′-dideoxyguanosine (ddG), 2′,3′-dideoxythymidine (ddT), 2′3′-deoxyinosine (ddI, e.g., didanosine), 2′,3′-didehydro-3′-deoxythimidine (d4T, e.g., stavudine), 2′,3′-dideoxydidehydrothymidine, 2′,3′-dideoxydidehydrocytidine, ganciclovir, acycloguanosine, fialuridine (FIAU), -2′,3′-dideoxy-3′-thiacytidine (3TC, e.g., lamivudine), lobucavir, (S)-i-[3-Hydroxy-2-(phosphonylmethoxy)propyl]cytosine (HPMPC, e.g., cidofovir), (R)-9-(2-phosphonylmethoxypropyl)adenine (PMPA) and the bis(isopropyloxycarbonyloxymethyl)-ester prodrug derivative of PMPA [bis(POC)-PMPA], abacivir (a.k.a. 1592U89), 9-(2-phosphonylmethoxyethyl)adenine (PMEA) and the bis(pivaloyloxymethyl)-ester produg derivative of PMEA [bis(POM)-PMEA, e.g., GS840 or adefovir dipivoxil], gemcitabine or combinations thereof.

In some embodiments of the invention, the insulin secreting cell is an immortalized cell line, and in some embodiments the insulin secreting cell is capable of being induced to differentiate and/or is undifferentiated.

One aspect of the invention provides a method of producing a cybrid cell line, comprising the steps of treating an insulin secreting cell line with an antiviral compound to convert the cell line into a ρ⁰ cell line, and then repopulating such a ρ⁰ cell line with isolated mitochondria to form a cybrid cell line. In one embodiment the cybrid cell line has extramitochondrial genomic DNA and mitochondrial DNA of differing biological origins. In a further embodiment the cybrid cell line has extramitochondrial genomic DNA and mitochondrial DNA from xenogeneic species. In a further embodiment the cybrid cell line has mitochondrial DNA from a rodent species, which may in further embodiments be mitochondrial DNA derived a mouse, rat, rabbit, hamster, guinea pig or gerbil. In one such further embodiment the cybrid cell line has mitochondrial DNA from a BHE/cdb rat.

It is another aspect of the invention to provide a method of producing a cybrid cell line, by treating an insulin secreting cell line with an antiviral compound to convert the cell line into a mitochondrial DNA depleted cell line, and then repopulating such a mitochondrial DNA depleted cell line with isolated mitochondria, to form the cybrid cell line. In certain embodiments of this aspect of the invention, the cybrid cell line has extramitochondrial genomic DNA and mitochondrial DNA of differing biological origins. In certain embodiments the cybrid cell line has extramitochondrial genomic DNA and mitochondrial DNA from xenogeneic species. In certain embodiments the cybrid cell line has mitochondrial DNA from a rodent species, which in certain further embodiments may be mitochondrial DNA from a mouse, rat, rabbit, hamster, guinea pig or gerbil. In one further embodiment the cybrid cell line has mitochondrial DNA from a BHE/cdb rat.

In certain embodiments of the invention, a cybrid cell line is produced by treating an insulin secreting cell line with an antiviral compound that is a nucleoside, nucleotide or base analog or a prodrug thereof. In some embodiments the antiviral compound may be 2′,3′-dideoxycytidine (ddC), 3′-azido-3′ deoxythymidine (AZT, e.g., zidovudine or ZDV), 2′,3′-dideoxyadenosine (ddA), 2′,3′-dideoxyguanosine (ddG), 2′,3′-dideoxythymidine (ddT), 2′3′-deoxyinosine (ddI, e.g., didanosine), 2′3′-didehydro-3′-deoxythimidine (d4T, e.g., stavudine), 2′,3′-dideoxydidehydrothymidine, 2′,3′-dideoxydidehydrocytidine, ganciclovir acycloguanosine, fialuridine (FIAU), -2′,3′-dideoxy-3′-thiacytidine (3TC, e.g., lamivudine), lobucavir, cidofovir (e.g., HPMPC), PMPA, abacivir (e.g., 1592U89), bis-POM PMEA (e.g., GS840 or adefovir dipivoxil), gemcitabine or combinations thereof.

In some embodiments of the invention, the insulin secreting cell line to be treated with an antiviral compound is an immortalized cell line. In certain embodiments the cybrid cell line produced according to the method provided is capable of secreting insulin. In certain embodiments the cybrid cell line produced according to the method provided is capable of responding to insulin. In certain embodiments the the cell line is derived from a pancreatic beta cell. In certain embodiments the cell line is an undifferentiated cell line that is capable of being induced to differentiate.

Some embodiments of the invention provide a method of producing a cybrid cell line using isolated mitochondria that are obtained from a subject known to be afflicted with a disorder associated with a mitochondrial defect. In some embodiments of the invention that provide methods of producing a cybrid cell line having extramitochondrial genomic DNA and mitochondrial DNA of differing biological origins, the extramitochondrial genomic DNA has its origin in an immortal cell line, and the mitochondrial DNA has its origin in a human tissue sample. In certain of these embodiments the human tissue sample is further derived from a patient having a disease that is associated with a mitochondrial defect.

It is another aspect of the present invention to provide a method of constructing an immortal cybrid cell line, comprising the steps of: treating an immortal insulin secreting cell line with an antiviral compound to convert the cell line into an immortal ρ⁰ cell line, and repopulating the immortal ρ⁰ cell line with mitochondria isolated from tissue of a patient afflicted with diabetes mellitus, Alzheimer's Disease, Parkinson's Disease, Huntington's disease, dystonia, Leber's hereditary optic neuropathy, schizophrenia, myoclonic epilepsy lactic acidosis and-stroke (MELAS), or myoclonic epilepsy ragged red fiber syndrome (MERRF), NARP (Neuropathy; Ataxia; Retinitis Pigmentosa), MNGIE (Myopathy and external ophthalmoplegia; Neuropathy; Gastro-Intestinal; Encephalopathy), Kearns-Sayre disease, Pearson's Syndrome, PEO (Progressive External Ophthalmoplegia); congenital muscular dystrophy with mitochondrial structural abnormalities, Wolfram syndrome (DIDMOAD, Diabetes Insipidus, Diabetes Mellitus, Optic Atrophy, Deafness), Leigh's Syndrome, fatal infantile myopathy with severe mtDNA depletion, benign “later-onset” myopathy with moderate reduction in mtDNA, dystonia, arthritis, and mitochondrial diabetes and deafness (MIDD), to form the cybrid cell line.

It is another aspect of the invention to provide a method of constructing an immortal cybrid cell line by treating an immortal insulin secreting cell line with an antiviral compound to convert the cell line into an immortal mitochondrial DNA depleted cell line; and repopulating such an immortal mitochondrial DNA depleted cell line with mitochondria isolated from tissue of a patient afflicted with diabetes mellitus, Alzheimer's Disease, Parkinson's Disease, Huntington's disease, dystonia, Leber's hereditary optic neuropathy, schizophrenia, myoclonic epilepsy lactic acidosis and stroke (MELAS), or myoclonic epilepsy ragged red fiber syndrome (MERRF), to form the cybrid cell line.

In another aspect of the invention, a method is provided for preparing a cybrid animal, by treating embryonic cells isolated from a multicellular, non-human animal with an antiviral compound to convert the cells to a ρ⁰ state, and then repopulating these ρ⁰ embryonic cells with mitochondria isolated from another cell source, to produce a cybrid animal.

In another aspect of the invention, a method is provided for preparing a cybrid animal, by treating embryonic cells isolated from a multicellular, non-human animal with an antiviral compound to convert the cells to a mitochondrial DNA depleted state, and then repopulating these mitochondrial DNA depleted embryonic cells with mitochondria isolated from another cell source, to produce a cybrid animal.

In another aspect, the invention provides a method of detecting a disease associated with altered mitochondrial function by treating an insulin secreting cell line with an antiviral compound to convert the cell line into a mitochondrial DNA depleted cell line or a ρ⁰ cell line, repopulating such a mitochondrial DNA depleted cell line or ρ⁰ cell line with mitochondria from a donor subject suspected of having a disease associated with altered mitochondrial function to produce a cybrid cell line, determining altered levels of insulin secretion by such a cybrid cell line and therefrom identifying the mitochondria donor subject as having a disease associated with altered mitochondrial function.

In another aspect, the invention provides a method of detecting a disease associated with altered mitochondrial function comprising treating an insulin secreting cell line with an antiviral compound to convert the cell line into a mitochondrial DNA depleted cell line or a ρ⁰ cell line, repopulating such a mitochondrial DNA depleted cell line or ρ⁰ cell line with mitochondria from a donor subject suspected of having a disease associated with altered mitochondrial function to produce a cybrid cell line, comparing altered levels of insulin secretion by such a cybrid cell line to insulin secretion by an insulin secreting cell line having mitochondria from a subject with normal mitochondrial function and therefrom identifying the mitochondria donor subject as having a disease associated with altered mitochondrial function.

In another aspect, the invention provides a method of evaluating an antiviral compound for its effect on mitochondrial function, by treating an insulin secreting cell line with an antiviral compound to convert the insulin secreting cell line into a mitochondrial DNA depleted cell line or a ρ⁰ cell line, repopulating the mitochondrial DNA depleted cell line or ρ⁰ cell line with mitochondria to produce a cybrid cell line, and determining insulin secretion by the cybrid cell line in the presence or absence of an antiviral compound, therefrom identifying an effect of the antiviral compound on mitochondrial function. In certain embodiments, the mitochondria are from a subject suspected of having a disease associated with altered mitochondrial function. In certain embodiments, the cybrid cell line has extramitochondrial genomic DNA and mitochondrial DNA of differing biological origins. In certain embodiments the cybrid cell line has extramitochondrial genomic DNA and mitochondrial DNA from xenogeneic species. In some embodiments the cybrid cell line has mitochondrial DNA from a rodent species. In some embodiments the cybrid cell line has mitochondrial DNA from a mouse, rat, rabbit, hamster, guinea pig or gerbil. In certain embodiments the cybrid cell line has mitochondrial DNA from a BHE/cdb rat.

In another aspect, the invention provides a method of identifying an agent that at least partially restores insulin secretion to a cell exposed to an antiviral compound which inhibits insulin secretion, comprising treating an insulin secreting cell line with an antiviral compound to convert the cell line into a mitochondrial DNA depleted cell line or a ρ⁰ cell line, repopulating such a mitochondrial DNA depleted cell line or ρ⁰ cell line with mitochondria to produce a cybrid cell line, contacting such a cybrid cell line with a candidate agent capable of at least partially restoring insulin secretion to the cybrid cell line, detecting an increase in insulin secretion by the cybrid cell line and therefrom identifying an agent that partially restores insulin secretion.

In another aspect, the invention provides a method for selecting a therapeutic agent suitable for use in a subject having a disease associated with altered mitochondrial function, comprising treating an insulin secreting cell line with an antiviral compound to convert the cell line into a mitochondrial DNA depleted cell line or a ρ⁰ cell line, repopulating such a mitochondrial DNA depleted cell line or ρ⁰ cell line with mitochondria from a subject having a disease associated with altered mitochondrial function to produce a cybrid cell line, detecting the level of insulin secretion by such cybrid cell line, contacting the cybrid cell line with a candidate therapeutic agent, detecting the effect of the candidate therapeutic agent on insulin secretion by the cybrid cell line and therefrom determining the suitability of the therapeutic agent.

In another aspect, the invention provides a method for selecting a suitable therapeutic agent for use in a subject having a disease associated with impaired insulin secretion, comprising treating an insulin secreting cell line with an antiviral compound to convert the cell line into a mitochondrial DNA depleted cell line or a ρ⁰ cell line, repopulating such a mitochondrial DNA depleted cell line or ρ⁰ cell line with mitochondria from a subject having a disease associated with impaired insulin secretion to produce a cybrid cell line, detecting the level of insulin secretion by the cybrid cell line, contacting the cybrid cell line with a candidate therapeutic agent, detecting the effect of the candidate therapeutic agent on insulin secretion by the cybrid cell line and therefrom determining the suitability of the therapeutic agent.

It is another aspect of the invention to provide a method of evaluating the suitability of an antiviral compound for use in treating a virally infected patient, including in certain embodiments a patient having a disease associated with impaired insulin secretion, comprising determining the amount of mitochondrial DNA in at least one insulin secreting cell before and after contacting a candidate antiviral compound with the insulin secreting cell, and therefrom determining the suitability of the antiviral compound for treating the patient.

Turning to another aspect, the invention provides a method of evaluating the suitability of an antiviral compound for use in treating a virally-infected patient, including in certain embodiments a patient having a disease associated with impaired insulin secretion, comprising determining the amounts of (i) mitochondrial DNA in, and (ii) insulin secreted by, at least one insulin secreting cell before and after contacting a candidate antiviral compound with the cell, and therefrom determining the suitability of the antiviral compound for treating the patient.

In another aspect, the invention provides a method of evaluating a modification to an antiviral compound to determine if the modification alters side effects associated with the antiviral compound, comprising: comparing a difference d for each of a first and second candidate agent, the candidate agent being selected from the group consisting of the antiviral compound and a candidate antiviral compound comprising the modification, using the formula:

d=m2−m1

wherein m1 is a ratio calculated using the formula:

m1=b/a, wherein a is the amount of mitochondrial DNA in a first cell population comprising insulin secreting cells before contacting the cells with the candidate agent, and b is the amount of mitochondrial DNA in the first cell population after contacting the cells with the candidate agent, and wherein m2 is a ratio calculated using the formula:

m2=e/c, wherein c is the amount of mitochondrial DNA in a second cell population comprising rho revertants of the insulin secreting cells before contacting the cells with the candidate agent, and e is the amount of mitochondrial DNA in the second cell population after contacting the cells with the candidate agent; and therefrom determining if the modification alters side effects associated with the antiviral compound.

In a further embodiment, the method comprises comparing a difference p for each of a first and second candidate agent, the candidate agent being selected from the group consisting of the antiviral compound and a candidate antiviral compound comprising the modification, using the formula:

p=q2−q1

wherein q1 is a ratio calculated using the formula:

q1=s/r, wherein r is the amount of insulin secreted by a first cell population comprising insulin secreting cells before contacting the cells with the candidate agent, and s is the amount of insulin secreted by the first cell population after contacting said cells with the candidate agent, and wherein q2 is a ratio calculated using the formula:

q2=u/t, wherein t is the amount of insulin secreted by a second cell population comprising rho revertants of the insulin secreting cells before contacting the cells with the candidate agent, and u is the amount of insulin secreted by the second cell population after contacting the cells with the candidate agent; and therefrom determining if the modification alters side effects associated with the antiviral compound. In certain embodiments, the antiviral compound is a nucleoside analog.

The model systems described herein offer outstanding opportunities to identify, probe and characterize defective mitochondrial genes and mutations thereof, to determine their cellular and metabolic phenotypes, and to assess the effects of various drugs and treatment regimens in vitro and in vivo. Because such cell-based model systems are observed to undergo phenotypic changes characteristic of the diseases to which they relate, they can also be used in methods of diagnosis. By using these same cell cultures and/or animal models according to the invention in screening assays, it is also possible to predict which of several possible drugs or therapies may be desirable for a particular patient.

These and other aspects of the invention will become more apparent by reference to the following detailed description of the invention and attached drawings.

### BRIEF DESCRIPTION OF THE DRAWINGS

FIG. 1 illustrates the effect of exposure to various concentrations of three representative antiviral compounds for seven days on the relative mtDNA content of INS-1 cells.

FIG. 2 illustrates the effect of exposure to a representative antiviral compound for 0-40 days on the mtDNA content of INS-1 cells.

FIG. 3 illustrates the effect of exposure to a representative antiviral compound for 40 days on basal and glucose-stimulated insulin secretion by INS-1 cells.

FIG. 4 shows the ability of untreated INS-1 cells (“INS-1”) and ddC-treated INS-1 cells that have undergone Rho reversion [“INS(ρ⁰)”] to consume oxygen after the addition of 1 uM KCN.

### DETAILED DESCRIPTION OF THE INVENTION

The present invention provides improved methods and compositions for depleting mitochondrial DNA (mtDNA) from cells, such as insulin-secreting cells and cells that are derived from pancreatic beta cells, to generate ρ⁰ cells and mtDNA depleted cells that are useful in the production of cybrid cells and animals. Mitochondrial DNA is depleted from insulin-secreting cells by contacting such cells with an antiviral compound. Depletion of mtDNA with antiviral compounds provides a rapid method for producing insulin secreting mitochondrial cybrid cell lines, which may be of further use in providing disease models for mitochondria associated diseases. For example, cybrid cell models of diabetes mellitus may be produced according to the methods of the present invention. Other disease models may also be produced, depending on whether mitochondria from healthy or diseased individuals are used to repopulate cells depleted of mtDNA by treatment with an antiviral compound. The invention further provides methods for preparing cybrid animals by depleting mtDNA from embryonic cells using antiviral compounds and repopulating such cells with mitochondria from a distinct cellular source.

As noted above, the invention provides methods for generating ρ⁰ and mtDNA depleted cells by contacting insulin-secreting cells with an antiviral compound. Insulin-secreting cells include any cells that, naturally or as a result of genetic engineering, are capable of exporting any product of an insulin gene to the extracellular environment. Methods for determining whether a cell is an insulin-secreting cell are well known and include procedures for detecting the presence of insulin or proinsulin in the extracellular milieu of a cell. Such methods further include methods for quantifying insulin secreted by an insulin-secreting cell. For example, a radioimmunoassay (RIA) using an antibody that specifically binds to insulin may be used to identify a cell as an insulin-secreting cell. Variations on RIA such as enzyme linked immunosorbent assays and immunoprecipitation analysis, and other assays for the presence of insulin or proinsulin in a cell conditioned medium are readily apparent to those familiar with the art, and may further include assays that measure insulin secretion by cells in the presence or absence of secretagogues such as glucose, KCl, amino acids, sulfonylureas, forskolin, glyceraldehyde, succinate or other agents that may increase or decrease insulin or proinsulin in a cell conditioned medium. Such methods may also be used to quantify the amount of insulin produced by or released from an insulin-secreting cell.

Although the cells suggested for certain embodiments herein are insulin secreting pancreatic beta cells or cell lines that maintain a normal pancreatic beta cell or insulin responsive phenotype, the present invention is not limited to the use of such cells but may also include the use of other cells or cell lines that naturally or as the result of generic engineering may secrete insulin or proinsulin, including cells that secrete insulin or proinsulin in a regulated fashion. Rat insulinoma INS-1 cells are preferred for some aspects of the invention. In addition to INS-1 and INS-2 cells (Asfari et al., Endocrinology 130:167-178, 1992), other suitable cells are cells such as, but not limited to, various murine pancreatic βTC cell lines such as βTC1, βTC3, βTC6 and βTC7 (Nagamatsu et al., Endrocrinology 130:748-754, 1992; Efrat et al., Diabetes 42:901-907, 1993; Knaack et al., Diabetes 43:1413-1417, 1994); hamster β cell lines such as HIT-T15 (Civelek et al., Biochem J. 315:1015-1019, 1996; Santerre et al., Proc. Natl. Acad. Sci. U.S.A. 78:4339-4343, 1981); rat insulinoma (RIN) cell lines such as RINm5f (Gadzar et al., Proc. Natl. Acad. Sci. U.S.A. 77:3519-3523, 1980); and murine pancreatic β cell lines such as MIN6 (Miyazaki et al., Endocrinology 127:126-132, 1997; Soejima et al., J. Biol. Chem. 271:26194-26199, 1996). HIT-T15 (ATCC Accession No. CRL-1777) and RIN-m (ATCC Accession No. CRL-2057) and subclones thereof, including RIN-14B (CRL-2059), RIN-5F (CRL-2058) and RIN-m5F (CRL-1 1605), are available from the American Type Culture Collection (ATCC), Manassas, Va. Other insulin secreting cell types that are useful in the present invention include cells that are derived from pancreatic beta cells, as well as freshly isolated islets of Langerhans or islet cells in primary culture.

The use of established, culture-adapted insulin secreting cell lines is preferred for use in the methods of the invention. However, primary culture cells such as insulin secreting cells obtained by explant or biopsy from an individual known or suspected of suffering from a mitochondria associated disease or from another individual, e.g., an unaffected close blood relative of a patient suffering from a mitochondria associated disorder, may be used to generate ρ⁰ and mtDNA depleted cells according to the present invention. This use of genetically related cells may have certain advantages for ruling out non-mitochondrial effects as causative of particular phenotypic traits in cybrid cells produced from such ρ⁰ cells.

Genetically altered cells, such as transfected cell lines that are insulin secreting cells as a consequence of having undergone genetic transfection, are also within the scope of cells that may be used in the present invention. Such genetically altered cells may be differentiated or undifferentiated, and may further be cells that secrete insulin in a regulated fashion. Transfection of cells with genes encoding gene products of interest such as insulin or proinsulin, and transfection of cells with genes that include regulatory elements such as, but not limited to, specifically inducible promoters, enhancers and/or transcription factor binding sites, are well known in the art. (See, e.g., Newgard et al., J. Lab. Clin. Med. 122:356-363, 1993; Hughes et al., Proc. Nat. Acad. Sci. USA 89:688-692, 1992.)

Although insulin secreting cells themselves may be used as a preferred model system for mitochondria associated disease, it may also be preferred to propagate cells capable of secreting insulin in an undifferentiated state and to induce lineage-specific differentiation prior to screening assays or diagnostic assays. Physical, biological and/or chemical agents capable of inducing differentiation in particular undifferentiated cell lines are known in the art and may be used. In any event, it is most preferred to use recipient cells that can be induced to differentiate by the addition of particular chemical (e.g., hormones, growth factors, transcription factors, etc.) or physical (e.g., temperature, exposure to radiation such as U.V. radiation, etc.) induction signals The present invention also provides immortal cell lines that are undifferentiated or partially differentiated, but that are capable of being induced to differentiate, and further provides fully differentiated cell lines. These cell lines have origins in immortalized beta cells or insulin-responsive cells (for example, βTC6, HIT-T15, RINmSf, βTC-1 and INS-1 cells). “Immortal” cell lines refers to cell lines that may be so designated by persons of ordinary skill in the art, or that may be capable of being passaged preferably an indefinite number of times, but not less than ten times, without significant phenotypic alteration.

As noted above, the present invention provides novel compositions and methods that permit rapid generation of ρ⁰ and mtDNA depleted cell lines using antiviral compounds. An antiviral compound may be any composition that interferes with a viral structure or a viral function. Such interference of viral structure or viral function can be assessed in vivo or in vitro. Examples of antiviral compounds include but need not be limited to nucleoside analogs, nucleotide analogs, nucleoside or nucleotide base analogs, nucleic acid constructs, peptides, proteins, protease inhibitors, small molecules, cytokines and other compounds having antiviral activity. Viral functions include but need not be limited to any viral binding, infection, replication, gene expression, genetic recombination, integration, nucleic acid synthesis or particle assembly events. Viral functions may also include endocytic, phagocytic, nucleolytic, proteolytic, lipolytic, hydrolytic, catalytic, or other regulatory events. In addition, suitable antiviral compositions include those compositions that are known in the art for their antiviral activities, for instance in treating HIV infection. Suitable nucleoside, nucleotide and base analogs, and prodrugs thereof, include 3′-azido-3′ deoxythymidine (AZT, also known as zidovudine or ZDV), 2′,3′-dideoxycytidine (ddC) 2′,3′-dideoxyadenosine (ddA), 2′,3′-dideoxyguanosine (ddG), 2′,3′-dideoxythymidine (ddT), 2′3′-deoxyinosine (ddI, also known as didanosine), 2′3′-didehydro-3′-deoxythimidine (d4T, also known as stavudine), 2′,3′-dideoxydidehydrothymidine, 2′,3′-dideoxydidehydrocytidine, ganciclovir, acycloguanosine, fialuridine (FIAU), -2′,3′-dideoxy-3′-thiacytidine (3TC, also known as lamivudine), lobucavir, cidofovir (also known as HPMPC), PMPA, abacivir (also known as 1592U89), bis-POM PMEA (also known as GS840 or adefovir dipivoxil), gemcitabine and other nucleoside, nucleotide and base analogs or combinations thereof. Other nucleoside analogs are known to those familiar with the art, including those found in Kulikowski, Pharm. World Sci. 16:127-138, 1994; Isono, Pharmac. Ther. 52:269-286, 1991; and Isono, Jl. Antibiotics 41:1711, 1988; all of which are hereby incorporated by reference in their entireties. Combinations of nucleoside, nucleotide and base analogs, and prodrugs thereof, are useful in therapeutic modalities (Maenza et al., Am. Fam. Physician 57:2789-2798, 1998) and may also be employed in the present invention. Nucleoside, nucleotide and base analogs may interfere with viral nucleic acid synthesis and replication, for example by becoming incorporated into DNA or RNA molecules complementary to viral sequences or by other mechanisms. The structures of nucleoside analogs may be non-permissive for further extension of nucleic acid strands into which the analogs have been incorporated.

Prodrug forms of antiviral compounds such as those provided above are also within the scope of the invention. As used herein, the term “prodrug” refers to any compound that releases, or is metabolized to, an active drug after administration to an animal and/or after cellular internalization. For instance, a nucleoside analog may act as a prodrug because it is taken up by cells and enzymatically phosphorylated, thereby being converted into the corresponding nucleotide analog or a di- or tri-phosphate form of the nucleotide analog. One or more of these phosphorylated forms of the nucleoside analog, for example, may be the most active form of the drug (Kang et al., Pharm. Res. 14:706-712, 1997). As another example, chemical modifications to an antiviral compound may result in improved pharmacokinetic properties, and intracellular enzymes may act to remove the modifications after cellular internalization (Kerr et al., J. Med. Chem. 35:1996-2001, 1992). For a review of nucleotide prodrugs, see Jones et al. (Antiviral Res. 27:1-17, 1995).

Without wishing to be bound by theory, another biological activity of antiviral compounds (including nucleoside analogs) may be their inhibition of mitochondrial DNA (mtDNA) replication. These compounds are believed to incorporate into newly synthesized mtDNA, and may also inhibit DNA polymerase gamma, a mitochondria-specific enzyme required for mtDNA replication. Regardless of whether these or other mechanisms underlie the usefulness of antiviral compounds for the generation of ρ⁰ cells, the present invention provides for the generation of ρ⁰ cells for the production of cybrid cells from any cell line or cultured cell type.

As described herein, ρ⁰ cells and mtDNA depleted cells may be generated by contacting cells, such as insulin secreting cells, with an antiviral compound. Although those conditions suitable for generating such cells will be evident to those skilled in the art, for any particular combination of insulin-secreting cell and antiviral compound, preferred culture conditions may be determined using various concentrations of the antiviral compounds and exposure of cells to antiviral compound(s) over various time periods. For example, by way of illustration and not limitation, human INS-1 insulinoma cells may become ρ⁰ cells after exposure to 25 μm ddC for 4-8 weeks in culture media supplemented with pyruvate, uridine and glucose. For other cell types or cell lines, specific concentrations of antiviral compounds and duration of exposure may be optimized using routine methodologies with which those skilled in the art will be familiar in order to generate ρ⁰ cells or mtDNA depleted cells. Mitochondrial DNA depletion may be readily determined using slot blot analysis or other methods known to those of ordinary skill in the art, including methods for quantifying mtDNA such as those provided herein.

“ρ⁰ cells” are cells essentially completely depleted of mtDNA, and therefore have no functional mitochondrial respiration/electron transport activity. Such absence of mitochondrial respiration may be established by demonstrating a lack of oxygen consumption by intact cells in the absence of glucose, and/or by demonstrating a lack of catalytic activity of electron transport chain enzyme complexes having subunits encoded by mtDNA, using methods well known in the art. (See, e.g., Miller et al., J. Neurochem. 67:1897-1907, 1996.) That cells have become ρ⁰ cells may be further established by demonstrating that no mtDNA sequences are detectable within the cells. For example, using standard techniques well known to those familiar with the art, cellular mtDNA content may be measured using slot blot analysis of 1 μg total cellular DNA probed with a mtDNA-specific oligonucleotide probe radiolabeled with, e.g, ³²P to a specific activity >900 Ci/gm. Under these conditions ρ⁰ cells yield no detectable hybridizing probe signal.

Alternatively, any other method known in the art for detecting the presence of mtDNA in a sample may be used which provides comparable sensitivity. Such alternative methods may include, by way of example and not limitation, assays based on the polymerase chain reaction (PCR), including quantitative real-time PCR (Q-RTPCR, see, e.g., Freeman et al., BioTechniques 26:112-125, 1999 for a review; see also, e.g., Ahmed et al., BioTechniques 26:290-300, 1999); Southern hybridization techniques (see Schatz et al., Section IV of Chapter 2 in: Short Protocols in Molecular Biology, 2nd Ed., Ausubel et al., eds., John Wiley & Sons, New York, N.Y., 1992, pages 2-24 to 2-30); assays based on nucleic acid hybridization, including hybridizations carried out on micro-formatted multiplex or matrix devices (e.g., DNA or RNA chips, filters and microarrays; see, e.g., Bains, Bio/Technology 10:757-758, 1992); assays of mtDNA that utilize dyes that bind preferentially to mtDNA in living cells (see, e.g., Poglazova et al., Mikrobiologiia 59:1024-1031, 1990); and assays based on the differential migration of linear nuclear DNA and supercoiled mtDNA in gradients such as, e.g., CsCl-ethidium bromide gradients (Welter et al., Mol. Biol. Rep. 13:117-120, 1988).

“Mitochondrial DNA depleted” cells (“mtDNA depleted cells”) are cells substantially but not completely depleted of functional mitochondria and/or mitochondrial DNA, by any method useful for this purpose. MtDNA depleted cells are preferably at least 80% depleted of mtDNA as measured using the slot blot assay described above for the determination of the presence of ρ⁰ cells, and more preferably at least 90% depleted of mtDNA. Most preferably, mtDNA depleted cells are depleted of >95% of their mtDNA.

Mitochondria to be transferred to construct model systems in accordance with the present invention may be isolated from virtually any tissue or cell source. Cell cultures of all types may potentially be used, as may cells from any tissue. However, fibroblasts, brain tissue, myoblasts and platelets are preferred sources of donor mitochondria. Platelets are the most preferred, in part because of their ready abundance, and their lack of nuclear DNA. This preference is not meant to constitute a limitation on the range of cell types that may be used as donor sources.

In the examples below, platelets have been isolated by an adaptation of the method of Chomyn (Am. J. Hum. Genet. 54:966-974, 1994). However, it is not necessary that this particular method be used. Other methods are easily substituted. For example, if nucleated cells are used, cell enucleation and isolation of mitochondria isolation can be performed as described by Chromyn et al., Mol. Cell. Biol. 11:2236-2244, 1991. Human tissue from an individual with a disorder known to be associated with a mitochondrial defect that segregates with late onset diabetes mellitus may be the source of donor mitochondrial DNA.

After preparation of mitochondria by isolation of platelets or enucleation of donor cells, the mitochondria may be transplanted into ρ⁰ cells or mtDNA depleted cells using any known technique for introducing an organelle into a recipient cell, including but not limited to polyethylene glycol (PEG) mediated cell membrane fusion, cell membrane permeabilization, cell-cytoplast fusion, virus mediated membrane fusion, liposome mediated fusion, particle mediated cellular uptake, microinjection or other methods known in the art. For example by way of illustration and not limitation, mitochondria donor cells (˜1×10⁷) are suspended in calcium-free Dulbecco's modified Eagle (DME) medium and mixed with ρ⁰ cells (˜0.5×10⁶) in a total volume of 2 ml for 5 minutes at room temperature. The cell mixture is pelleted by centrifugation and resuspended in 150 μl PEG (PEG 1000, J.T. Baker, Inc., 50% w/v in DME). After 1.5 minutes, the cell suspension is diluted with normal ρ⁰ cell medium containing pyruvate, uridine and glucose, and maintained in tissue culture plates. Medium is replenished daily, and after one week medium lacking pyruvate and uridine is used to inhibit growth of unfused ρ⁰ cells. These or other methods known in the art may be employed to produce cytoplasmic hybrid, or “cybrid”, cell lines.

The present invention also provides insulin-responsive and insulin-secreting cybrid cell lines. In one embodiment of the invention, ρ⁰ cells generated from any insulin-secreting cell (e.g., derived from a human or non-human species) according to the method of the invention may be used to construct cybrid cells using mitochondria derived from a diabetic human or animal, for example a NIDDM patient, a BHE/cdb rat or other donor exhibiting impaired insulin secretion. Such cybrid cells may be used to screen for drug candidates able to reverse or minimize defects responsible for impaired insulin secretion in NIDDM.

Another embodiment of the invention provides ρ⁰ cells generated using the compositions and methods of the invention for construction of isogeneic or xenogeneic cybrid cells, (i.e., cybrid cells may be constructed having extramitochondrial genomic DNA and mtDNA that may be from the same species or that may be from different species). As a non-limiting example, cybrid cells may comprise human host cells and mitochondria from an animal model system. As another non-limiting example, donor mitochondria may be provided by platelets of the BHE/cdb rat, which expresses a mutation in the mitochondrial DNA-encoded ATP synthase 6 gene, and which develops a NIDDM-like syndrome (Kim et al., 1998 Int. J. Diabetes 6:1-11; Berdanier et al., 1997 Int. J. Diabetes 5:27-37; Berndanier, FASEB J. 5:2139-2144, 1991). As a further non-limiting example, donor mitochondria may be provided by a BHE/cdb rat and ρ⁰ cells for construction of cybrid cell lines may be derived from a rodent species, which may be, for instance, a mouse, rat, rabbit, hamster, guinea pig or gerbil.

In a preferred embodiment, the present invention provides the ability to model the precise genetic and biochemical defects in the NIDDM pancreas by providing insulin-secreting cell lines deficient in mitochondrial DNA. More particularly, the present invention provides an in vitro NIDDM model wherein depletion of mitochondrial DNA is associated with loss of glucose-stimulated insulin secretion. Cybrids may be constructed by repopulation of such mitochondrially depleted (ρ⁰) cells with mitochondria from normal or diseased (i.e., NIDDM) individuals. These cybrids may then be tested for restoration of glucose-stimulated insulin secretion. In a further embodiment, these cybrid cells produced from ρ⁰ cells generated according to the present invention may be screened for specific mitochondrial DNA mutations that may cause NIDDM. In another embodiment, these cybrid cells produced using mitochondria from NIDDM patients and exhibiting impaired insulin secretion may be used to screen for drug candidates that restore normal glucose-stimulated insulin secretion. In yet another embodiment, such cybrid cells may be used to screen for drug candidates that specifically reverse or minimize other biochemical and bioenergetic deficiencies that result from defects in NIDDM donor mitochondria.

In still another embodiment, the rapid generation of ρ⁰ cells that is made possible using the compositions and methods of the present invention permits construction of short-term cybrid cells, for example cybrid cells having mitochondria from NIDDM donors. Such short-term cybrids may not need to undergo transcription of mitochondrial DNA or mitochondrial replication to be useful. Instead, these cybrids can be promptly assayed for their glucose-stimulated insulin secretory responses or other phenotypic changes that may result from repopulation with potentially defective donor mitochondria.

Short-term cybrids as described above, or longer-term cybrids including cybrid cell lines, may be constructed in this manner using human ρ⁰ cells. Alternatively, isogeneic or xenogeneic cybrid cells may be produced using animal ρ⁰ cells and donor mitochondria from the same or different species. Where stable xenogeneic NIDDM cybrid cell lines are desired, po insulin secreting cells may be transfected with suitable genes for transcription and replication of donor mitochondrial DNA. It is known that a species-specific mitochondrial transcription factor and mitochondrial DNA polymerase y are required for transcription and replication of mitochondrial DNA, respectively (Clayton, Trends in Bioch. Sci. 16:107-111; Clayton, Int. Rev. Cytol. 141:217-232, 1992). It is further within the knowledge of one skilled in the art to stably transfect genes encoding mitochondrial transcription factor and DNA polymerase (into a cell that may be used to generate ρ⁰ cells for production of cybrid cell lines. For example, transformation of INS-1 insulinoma cells with donor-species genes encoding one or both of these factors may permit transcription of the donor mitochondrial genome.

In another embodiment, the invention provides a method for preparing a cybrid animal from ρ⁰ or mtDNA depleted embryonic cells generated using an antiviral compound according to the instant disclosure. For example by way of illustration and not limitation, mtDNA or mitochondria from a distinct biological source, such as a subject suspected of carrying a mitochondria associated disease, may be introduced into animals, creating a mosaic cybrid animal. As a further non-limiting example, a freshly fertilized mouse embryo, at about the 2 to 16 cell stage, may be washed by saline lavage from the fallopian tubes of a pregnant mouse. Under a dissection microscope, the individual cells may be teased apart, and treated with an antiviral compound, which may include a nucleoside analog, to induce a ρ⁰ state. Determining the appropriate duration and concentration for treatment with an antiviral compound may require the sacrifice of several embryos for Southern analysis to assure that mitochondrial function has been lost. Then, cells so treated may be repopulated with exogenous mitochondria isolated from a distinct biological source. One or more of the resulting cybrid cells may then be implanted into the uterus of a pseudopregnant female by microinjection into the fallopian tubes. At the end of gestation, the structure and/or activity of a mitochondrial gene in blood cells from one or more of the progeny may be tested to confirm that some of the mitochondria are derived from the donor. The presence of the donor mitochondrial DNA may also be confirmed by DNA sequence analysis.

Model systems made and used according to the present invention may be equally useful irrespective of whether the disease of interest is known to be caused by mitochondrial defects. Where mitochondrial disorders are a symptom of the disease, are associated with a predisposition to the disease, or have an unknown relationship to the disease, the present invention permits development of biological model systems that may be useful for screening assays to identify therapeutics or for diagnostic assays. In addition, the uses of model systems according to the present invention to determine whether a disease has an associated mitochondrial defect are within the scope of the present invention.

As a non-limiting example, the invention provides a method of detecting a disease associated with altered mitochondrial function by determining altered levels of insulin secretion by a cybrid cell line produced according to the methods disclosed herein, where such a cybrid cell line may contain mitochondria from a donor subject suspected of having a disease associated with altered mitochondrial function. Altered levels of insulin secretion, such as quantitative and/or qualitative (e.g., processing, posttranslational modification, cofactor requirements, etc.) differences in insulin secretion that may correlate with the introduction into these cells of mitochondria exhibiting altered function, may provide useful diagnostic information. Evaluation of potential mitochondria associated disease may further encompass quantitative and/or qualitative comparison of insulin secretion by a cybrid cell line that contains mitochondria from a donor subject suspected of having a disease associated with altered mitochondrial function, with insulin secretion by cybrid cells having normal mitochondria. These and similar uses of model systems according to the invention for the detection of diseases associated with altered mitochondrial function will be appreciated by those familiar with the art and are within the scope and spirit of the invention.

Model systems made and used according to the present invention may also be useful in the evaluation of antiviral compounds for their potential effects on mitochondrial function, which may further include the effect an antiviral compound may have on insulin secretion by a cell. For example by way of illustration and not limitation, the determination that an antiviral compound alters insulin secretion by an insulin secreting cybrid cell produced according to methods disclosed herein may be useful in the selection of antiviral compounds for therapeutic use in diseases, including but not limited to mitochondria associated diseases, in which altered mitochondrial function may be present as a result of the disease and/or as a consequence of any agent administered in the course of therapeutic treatment of the disease. As another example, evaluating the effect of a candidate therapeutic agent on insulin secretion by a cybrid cell produced according to the methods of the present invention may provide a method for selecting appropriate therapeutic agents for use in a subject having a disease associated with altered mitochondrial function, such as NIDDM. Accordingly, candidate therapeutic agents may be selected for their ability directly or indirectly to potentiate or impair insulin secretion.

As a further non-limiting example, model systems made and used according to the present invention may be useful for identifying agents that partially or completely restore insulin secretion to a cell exposed to an antiviral compound that inhibits insulin secretion. According to this example, impaired insulin secretion may be detected in an insulin secreting cybrid cell line produced as disclosed herein, and such an insulin secretion impaired cybrid cell line may be used to screen candidate agents by identifying those agents capable of effecting an increase in insulin secretion relative to the insulin secretion impaired state. In addition, the present invention provides model systems for selecting therapeutic agents that may be suitable for the treatment of diseases associated with altered mitochondrial function. These and similar uses of model systems according to the invention for the screening and identification of agents that counteract the effects an antiviral compound may exert on mitochondrial function, including insulin secretion, will be appreciated by those familiar with the art and are within the scope and spirit of the invention.

In certain embodiments, the invention provides a method of evaluating an antiviral compound, which may be a nucleoside analog, for its suitability for use in treating a disease associated with impaired insulin secretion, comprising contacting the antiviral compound with insulin secreting cells and determining the amount of mitochondrial DNA in the insulin secreting cells before and after being contacted with the antiviral compound. For example, a decreasing amount of mitochondrial DNA in the insulin secreting cells after being contacted with the antiviral compound may correspond to the antiviral compound having decreased suitability for use in treating a disease associated with impaired insulin secretion. In a related aspect, the invention provides a method of evaluating an antiviral compound for its suitability for use in treating a disease associated with impaired insulin secretion, comprising contacting the antiviral compound with insulin secreting cells, determining the amount of mitochondrial DNA in the insulin secreting cells before and after being contacted with the antiviral compound and also determining the amount of insulin secreted by the cells before and after being contacted with the antiviral compound. For example, a decreasing amount of mitochondrial DNA in the insulin secreting cells after being contacted with the antiviral compound and a decreasing amount of insulin secretion by the insulin secreting cells after being contacted with the antiviral compound may correspond to the antiviral compound having decreased suitability for use in treating a disease associated with impaired insulin secretion.

In certain other embodiments, the invention provides biological models and methods for evaluating a modification to an antiviral compound to determine if the modification ameriolates or exacerbates undesirable side-effects associated with the antiviral compound. Such a modification may be a chemical modification to the antiviral compound, or a modification to or change of the vehicle used to deliver the antiviral compound to cells, or both. One such method comprises (i) contacting an antiviral compound comprising the modification with a first cell population of insulin secreting cells and a second cell population composed of Rho revertants of the insulin secreting cells, (ii) determining the amount of mitochondrial DNA in the first cell population before and after the step of contacting, and calculating therefrom the ratio:

m1=b/a,

which ratio represents the amount of mitochondrial DNA in the first cell population after the first cell population is contacted with the modified antiviral compound (b), relative to the amount of mtDNA before such contact (a); (iii) determining the amount of mitochondrial DNA in the second cell population before (c) and after (e) the second cell population is contacted with the modified antiviral compound to calculate therefrom the ratio:

m2=e/c,

and (iv) determining the difference (d):

d=m2−ml.

For example, a decrease in the value d calculated for a modified antiviral compound relative to the value for d calculated for the unmodified antiviral compound may indicate that the modification ameliorates undesirable side effects associated with the antiviral compound. Conversely, an increase in the value for d may indicate that the modification exacerbates such undesirable side effects. Thus, for instance, where the value of m2 may be about 1, the amount of mitochondrial DNA in the second cell population comprising rho revertants is not significantly affected by treatment with the candidate agent. In this instance, if d is a negative number, then the candidate agent causes a decrease in the amount of mitochondrial DNA in the first (non-revertant) cell population, but not in the second cell population comprising rho revertants. Alternatively, in this instance, if d is about 0.00, then the candidate agent does not significantly alter the amount of mitochondrial DNA in the first (non-revertant) cell population relative to the second cell population comprising rho revertants. Modifications that yield a value of d that is about 0.00 thus have less impact on mitochondrial DNA and are expected to be less toxic to patients.

In a related further embodiment, the first and second cell populations are also tested for the amount of insulin they secrete before and after the cell populations are contacted with the modified antiviral compound. Accordingly, the method may further comprise (v) determining the amount of insulin secreted in the first cell population before and after the step of contacting, and calculating therefrom the ratio:

q1=s/r,

which ratio represents the amount of insulin secreted by the first cell population after the first cell population is contacted with the modified antiviral compound (s), relative to the amount of insulin secreted before such contact (r); (vi) determining the amount of insulin secreted by the second cell population before (t) and after (u) the second cell population is contacted with the modified antiviral compound to calculate therefrom the ratio:

q2=u/t,

and (vii) determining the difference (p):

p=q2−q1.

For example, a decrease in the value p calculated for a modified antiviral compound relative to the value for p calculated for the unmodified antiviral compound may indicate that the modification ameliorates undesirable side effects associated with the antiviral compound. Conversely, an increase in the value for p may indicate that the modification exacerbates such undesirable side effects. Thus, for instance, where the value for q2 may be about 1, the amount of insulin secreted by the second cell population comprising rho revertants is not significantly affected by treatment with the candidate agent. In this instance, if p is a negative number, then the candidate agent causes a decrease in the amount of insulin secreted by the first (non-revertant) cell population relative to the comparably treated second cell population comprising rho revertants. Alternatively, in this instance, if p is about 0.00, then the candidate agent does not significantly alter the amount of insulin secreted by the first (non-revertant) cell population relative to the second cell population comprising rho revertants. Modifications that yield a value of p that is about 0.00 thus have less impact on insulin secretion and are expected to cause less side-effects in patients. In certain embodiments, the patients have a disease associated with impaired insulin secretion. These and related aspects and embodiments of the invention will be appreciated by those familiar with the art, and are within the scope of the present invention.

In addition, although the present invention is directed primarily towards model systems for diseases in which the mitochondria have metabolic defects, it is not so limited. Conceivably there are disorders wherein mitochondria contain structural or morphological defects or anomalies, and the model systems of the present invention are of value, for example, to find drugs that can address that particular aspect of the disease. In addition, there are certain individuals that have or are suspected of having extraordinarily effective or efficient mitochondrial function, and the model systems of the present invention may be of value in studying such mitochondria. In addition, it may be desirable to put known normal mitochondria into cell lines having disease characteristics, in order to rule out the possibility that mitochondrial defects contribute to pathogenesis. All of these and similar uses are within the scope of the present invention, and the use of the phrase “mitochondrial defect” herein should not be construed to exclude such embodiments.

It is important to an understanding of the present invention to note that all technical and scientific terms used herein, unless otherwise defined, are intended to have the same meaning as commonly understood by one of ordinary skill in the art. The techniques employed herein are also those that are known to one of ordinary skill in the art, unless stated otherwise. Throughout this application various publications are referenced within parentheses. The disclosures of these publications in their entireties are hereby incorporated by reference in this application.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed.

The following examples are offered by way of illustration and not limitation, and are not intended to limit the scope and spirit of the invention that shall be apparent to those having skill in the art.

### EXAMPLES

### Example 1

### Treatment of Cells With Nucleoside Analogs to Deplete Mitochondrial DNA

Cell Culture

INS-1 rat insulinoma cells were provided by Prof. Claes Wollheim, University Medical Centre, Geneva, Switzerland, and cultured at 37° C. in a humidified 5% CO₂ environment in RPMI cell culture media (Gibco BRL, Gaithersburg, MD) supplemented with 10% fetal bovine serum (Irvine Scientific), 2 mM L-glutamine, 100 U/ml penicillin, 100 μg/ml streptomycin, 10 mM HEPES, 1 mM sodium pyruvate and 50 μM β-mercaptoethanol.

Generation of ρ⁰ Cells

INS-1 cells were cultured for 3-60 days under conditions as described above except media were additionally supplemented with 50 μg/ml uridine and nucleoside analogs 2′3′-dideoxycytidine [ddC], 2′3′-dideoxyinosine [ddI] or 2′3′-didehydro-3-deoxythymidine [d4T] (all from Sigma) at varying concentrations (1-500 μM) diluted from 100× stock in PBS or a comparable dilution of PBS without. Media were replenished every two days. Cells were harvested at periodic intervals and assayed for insulin secretion and mtDNA content.

### Example 2

### Depletion of Mitochondrial DNA in Cells Treated With Nucleoside Analogs

DNA Probes

Total DNA was prepared from rat liver (for probing rat-derived cells) or the murine cell line 3T3 L1 (for probing mouse-derived cells; see Green et al., Cell 3:127-133, 1974 and Cell 5:19-27, 1975) using DNAzolTm reagents (Molecular Research Center, Inc., Cincinnati, Ohio) and method essentially according to the manufacturer's instructions. The template DNAs were examined by agarose gel electrophoresis and ethidium bromide staining and found to be roughly equivalent. Each template DNA was used in separate polymerase chain reaction (PCR) reactions to prepare DNA molecules having 1,207 base pairs and corresponding to either nucleotides 5342 to 6549 of the rat (Rattus norvegicus) mitochondrial genome (GenBank Accession No. X14848, Anderson et al., Nature 290:497-516, 1981) or nucleotides 5361 to 6568 of the murine (Mus musculus) mitochondrial genome (GenBank Accession No. V0071 1, Bibb et al., Cell 26:167-180, 1981). The same pair of oligonucleotide primers, specific for the mitochondrially encoded cytochrome c oxidase subunit I (COX-I) gene, were used for reactions for either rat or mouse templates. The pair of primers consisted of forward and reverse oligonucleotides having the following sequences:

| | | |
|---|---|---|
| Forward: | 5′-CACAAAGATATCGGAACCCTCTA | (SEQ ID NO:1) |
| Reverse: | 5′-AAGTGGGCTTTTGCTCATGTGTCAT | (SEQ ID NO:2) |

The PCR reactions contained appropriate amounts of template DNA, primers, MgCl₂, all four dNTPs, reaction buffer, and Taq polymerase, brought up to a volume of 50 ul using sterile water. The reactions were incubated at 95° C. for 10 seconds, followed by 30 cycles of 95° C. for 1 minute, 60° C. for 1 minute and 72° C. for 1 minute, after which the reactions were incubated at 72° C. for 4 minutes and then cooled to 4° C.

The PCR reactions mixes were extracted with phenol:chloroform and, along with a series of molecular weight markers, electrophoresed on an agarose gel that was stained with ethidium bromide and visualized with ultraviolet light. For both reactions, a single band of the predicted size (i.e., about 1.2 kilobases) was observed. The rat probe was radiolabeled with ³²P using a Prime-a-Gene® random priming kit (Promega, Madison, Wis.) essentially according to the manufacturer's instructions.

Quantification of Mitochondrial DNA by Slot Blotting

INS-1 cells, or ρ⁰ INS-1 cells generated using ddC as described above, were seeded into 12-well plates containing RPMI media supplemented as described above at 0.4×10⁶ cells/well and cultured at 37° C., 5% CO₂ for 2 days. Cells (0.7×10⁶ cells/well) were rinsed with PBS and total cellular DNA was extracted using DNAzol (Molecular Research Center, Inc., Cincinnati, Ohio) according to the manufacturer's instructions. One hundred ng DNA from each cell preparation was slot-blotted onto a Zeta-Probe membrane (Bio-Rad, Hercules, Calif.) and crosslinked at 125 joules using a BioRad GS GeneLinker irradiation/energy source.

The membranes were rinsed in hybridization buffer (5× SSC, 0.1% N-laurylsarcosine, 0.02% SDS, 1% blocking solution, Boehringer Mannheim, Indianapolis, Ind.) and hybridized overnight in the same buffer at 42° C. with the [³²P]-labeled rat COX I probe. Following hybridization, membranes were washed twice with 2X SSC/0.1% SDS and twice with 0.1× SSC/0.1% SDS and exposed to X-ray film. Mitochondrial DNA was quantified by densitometric scanning of the resulting autoradiographs.

Results

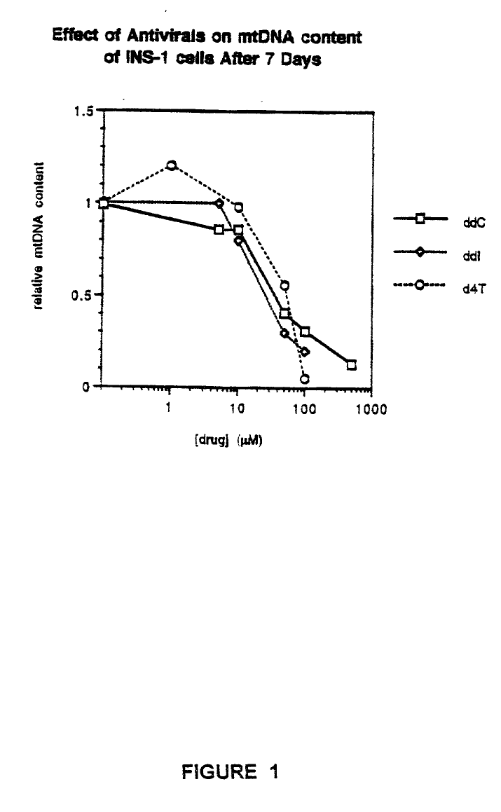

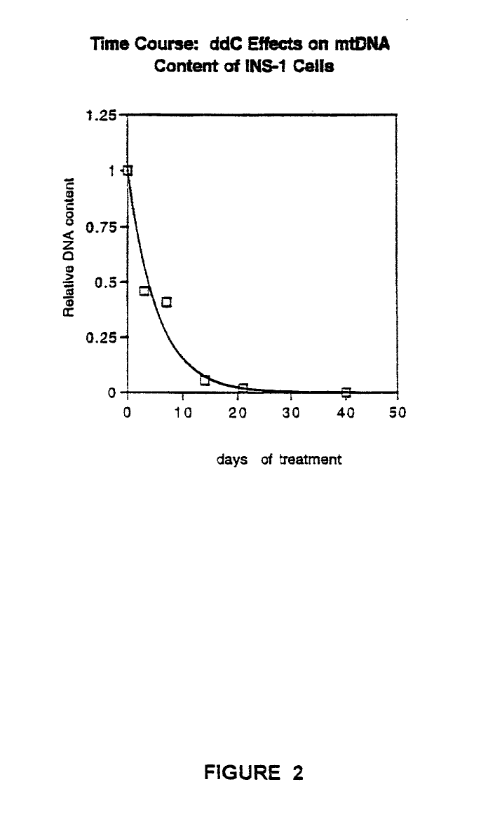

Incubation of INS-1 cells with ddC, ddI or d4T for seven days decreased mtDNA content in a dose-dependent fashion, as shown in FIG. 1. The relative mtDNA content (mean COX-I hybridization signal+SEM) of the cells, normalized to total cellular DNA, is plotted as a function of nucleoside analog concentration. The IC₅₀ for ddC was approximately 50 μM. In INS-1 cells incubated with 25 μM ddC for up to 40 days, the decline in mtDNA content was time-dependent, with a t_{½} of approximately three days; mtDNA was undetectable in these cells after 21 days. (FIG. 2.) AZT and FIAU were evaluated for their ability to deplete cells of mitochondrial DNA in the following experiment. INS-1 cells were grown and contacted with nucleoside analogs at varying concentrations as described in Example 1 for seven days, and mtDNA content was determined by slot blot analysis as described infra. A dye, 2-[4(dimethylamino)styryl]methylpyridinium iodide (2-4-DSM), known to accumulate in mitochondria in a membrane potential dependent manner (Morozova et al., Tsitologiia 23:916-923, 1981), was used as a positive control.

The results are presented in Table 1. As in the results shown in FIG. 1, the antiviral nucleoside analogs ddC, ddI and d4T each cause a level of dose-dependent depletion of mitochondrial DNA that is comparable to that caused by 2-4-DSM. That is, when cells are incubated in the presence of ddC, ddI or d4T at a concentration of 100 μM for seven days, the cells are respectively 69%, 80% and 95% depleted for mitochondrial DNA. The activity of AZT is somewhat more variable in this assay: treatment of cells with AZT for seven days at concentrations of 10 and 50 μM results in cells that are 4% and 15% depleted for mtDNA, whereas no depletion of mtDNA is seen over this period of time when the concentration of AZT is 5 or 100 μM. This may reflect a more frequent incidence of rho revertant cells (see Example 4, infra) when INS-1 cells are treated with AZT. Treatment with FIAU at the same concentration for the same period of time results in cells that are 61% depleted for mtDNA.

TABLE 1
RELATIVE AMOUNTS OF MITOCHONDRIAL DNA IN INS-1 CELLS
TREATED WITH ANTIVIRAL NUCLEOSIDE ANALOGS FOR SEVEN DAYS

| Anti- | | | | | | | |
|---|---|---|---|---|---|---|---|
| viral | | | | | | | 2-4-DSM |
| Conc. | Relative | [mtDNA] | After | Contact | w/ 2-4-DSM or | Antiviral | Conc |
| (μM) | ddC | ddI | d4T | AZT | FIAU | 2-4-DSM | (μg/ml) |
| 0 | 0.99 | 1.00 | 1.00 | 1.00 | 1.00 | 1.00 | 0.0 |
| 5 | 0.86 | 1.00 | (N.D.) | 1.09 | (N.D.) | 0.90 | 0.5 |
| 10 | 0.86 | 0.80 | 0.98 | 0.96 | 0.66 | 0.83 | 1.0 |
| 50 | 0.41 | 0.30 | 0.56 | 0.81 | (N.D.) | 0.36 | 5.0 |
| 100 | 0.31 | 0.20 | 0.05 | 1.03 | 0.39 | (N.D.) | — |

Notes:
1. “2-4-DSM” is 2-[4(dimethylamino)styryl]methylpyridinium iodide.
2. “N.D.”, not determined.

### Example 3

### Insulin Secretion by Mitochondrial DNA-depleted Cells Generated Using Nucleoside Analogs

INS-1 cells, or ρ⁰ INS-1 cells generated using ddC as described above, were seeded into 12-well plates containing RPMI media supplemented as described at 0.5×10⁶ cells/well and cultured at 37° C., 5% CO₂ for 2 days. Cells (0.7×10⁶ cells/well were rinsed with glucose-free KRH buffer (134 mM NaCl, 4.7 mM KCl, 1.2 mM KH₂PO4, 1.2 mM MgSO₄, 1.0 mM CaCl₂, 10 mM HEPES, 10 mM NaHCO₃, 0.5% BSA), then incubated in the same buffer for 1 hr at 37° C. in a humidified 5% Co₂/95% air atmosphere. Fresh KRH buffer containing 0.5 mM isobutylmethyl xanthine and the following secretagogues was added: 5 mM glucose, 10 mM glucose, 20 mM glucose, 5 mM KCl or 20 mM KCl. After an additional 1 hr at 37° C., 5% CO₂ the culture supernatants were collected. Insulin concentrations in the supernatants were measured and normalized to cell number using an insulin-specific radioimmunoassay kit (ICN Biochemicals, Irvine, CA) according to the manufacturer's instructions.

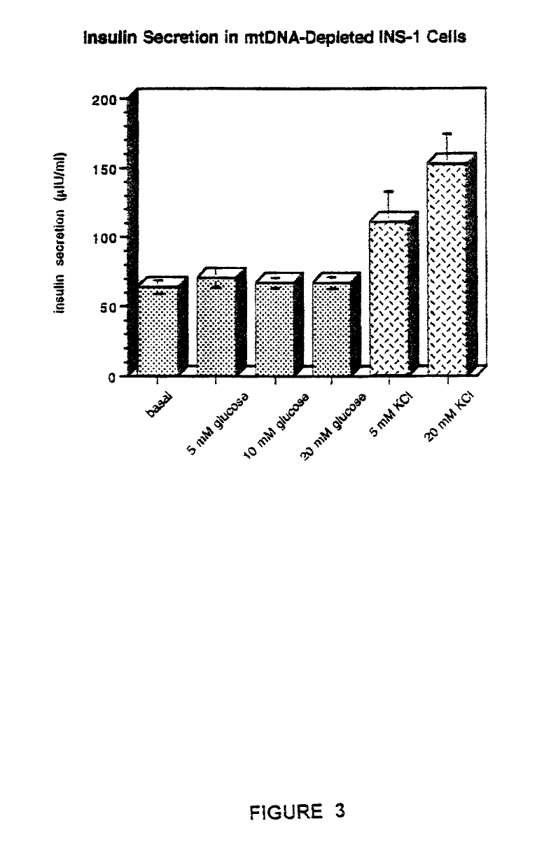

INS-1 cells normally exhibit half-maximal glucose-mediated insulin secretion at 5 mM glucose. Following treatment with ddC (10 μM) for 40 days, at which time mtDNA was undetectable, no glucose stimulated insulin secretion was observed at any glucose level tested (FIG. 3). In contrast, KCl-mediated insulin secretion, which bypasses the mitochondrial component of the insulin secretory pathway, remained intact.

### Example 4

### Reversion of Cells Treated with Antiviral Agents

When treated with antiviral agents such as dideoxycytidine (ddC), some or all of the cells are able to “escape” the mtDNA-toxic effects thereof and apparently restore, or become “repleted” with, mtDNA. A variety of genetic and/or biochemical events may account for the emergence of these revertant cells. The addition of more than one antiviral agent, or the substitution of a second antiviral agent for the first agent during antiviral treatment, is expected to prevent the development of such revertant cells. In any event, because revertant cells do not develop until after about 40 days or longer of antiviral treatment, experiments can be performed on mtDNA depleted cells prior to the appearance of rho revertants. The timing of the appearance of rho revertants can vary among cell lines and as a function of a particular cell type and/or a particular antiviral agent. Based on the disclosure herein, a person having ordinary skill in the art can readily determine the presence of rho revertants in a population of cells such as those described below, or in any other suitable cell population.

Time Course of Effects of ddC on Mitochondrial DNA Content of INS-1 Cells INS-1 cells were grown in RPMI medium supplemented with 10% fetal calf serum, 2 mM 1-glutamine, 100 U/ml penicillin, 100 μg/ml streptomycin, 10 mM HEPES, 1 mM sodium pyruvate, 50 μg/ml uridine, and 50 μM β-mercaptoethanol, in the presence of 10 μM ddC. After various times in culture, cells were harvested for analysis of mtDNA content. Total cellular DNA was extracted from approximately one million cells using DNAzol (Molecular Research Center, Inc. Cincinnati, Ohio) essentially according to the manufacturer's instructions. One hundred ng total DNA was analyzed by slot-blot on Zeta-Probe membranes (Bio-Rad, Hercules, Calif.). The membranes were hybridized with a [³²P]-labeled probe complementary to the mitochondrial gene encoding COXI. Autoradiograms were prepared, and the relative content of mitochondrial DNA (mtDNA) estimated by the relative densities of the autoradiographic bands.

The results (Table 2) show that the cells become essentially mitochondrial DNA depleted by day 14 and are severely mtDNA depleted by day 40. However, even though the ddC treatment was maintained throughout the experiment described in Table 2, the mtDNA content of the cellular population began to rise sometime between day 40 and day 60. The mtDNA content of about one million cells prepared at day 80 was about 80% that of about one million cells prepared pretreatment (day 0).

TABLE 2
RELATIVE MTDNA CONTENT IN CELLS TREATED WITH
DDC OVER TIME

| Day Number | Relative mtDNA content per million cells | Standard Error |
|---|---|---|
| 0 | 1.00 | 0.00 |
| 3 | 0.46 | 0.00 |
| 7 | 0.41 | 0.18 |
| 14 | 0.055 | 0.025 |
| 21 | 0.020 | 0.00 |
| 40 | 0.0021 | |
| 60 | 0.550 | |
| 80 | 0.81 | |

Rho Reversion in Antiviral Treated Cells

Rho reversion of ddC-treated cells was evaluated by assaying mitochondrial biochemical activities using methods known in the art. Any of a variety of such activities may be measured to monitor phenotypic aspects of Rho reversion; in the interest of brevity, results of the evaluation of rho reversion by assaying two representative mitochondrial biochemical activities are presented here.

1. Lactate Production

In rho⁰ (ρ⁰) INS-1 cells, lactate production is greatly increased due to inability of the cells to metabolize endproducts of glycolysis mitochondrially. Lactate production was measured in ddC-treated and control INS-1 cells at approximately day 40 and day 50 of ddC incubation. Cells were grown in 35 mm dishes. Media were replenished 16 hr before assay with normal culture media containing various amounts of glucose. The media were then collected, and lactate measured using a commercially available kit, in which lactate dehydrogenase is used to produce a fluorescent compound (Sigma, St. Louis, Mo.), essentially according to the manufacturer's instructions.

The results are presented in Table 3. At 40 days, the control (untreated) cells accumulated only small amounts of lactate, whereas, when supplied 25 or 50 mM glucose, the 40-day ddC-treated, mtDNA depleted cells produced >5 times as much lactate as the control cells. In contrast, after more than about 50 days, the control (untreated) cells and ddC-treated cells produced comparable amounts of lactate. These results indicate that the collection of ddC-treated cells had, after about day 50, reverted to a rho+(i.e., mtDNA+) state.

2. Oxygen Consumption

By definition, rho⁰ cells do not consume oxygen when provided with glucose as substrate. Control INS-1 cells and INS-1 cells that had been treated with ddC for >50 days (ρ⁰ revertants) were harvested, suspended in Hank's balanced salt solution at a concentration of 10 million cells per ml, and analyzed using a Clark oxygen electrode and monitor (Yellow Springs, Yellow Springs, Ohio; see Miller et al., J. Neurochem. 67:1897-1907, 1996) before and after the addition of 1 uM KCN. KCN inhibits mitochondrial respiration, primarily via its effect on Complex IV activity.

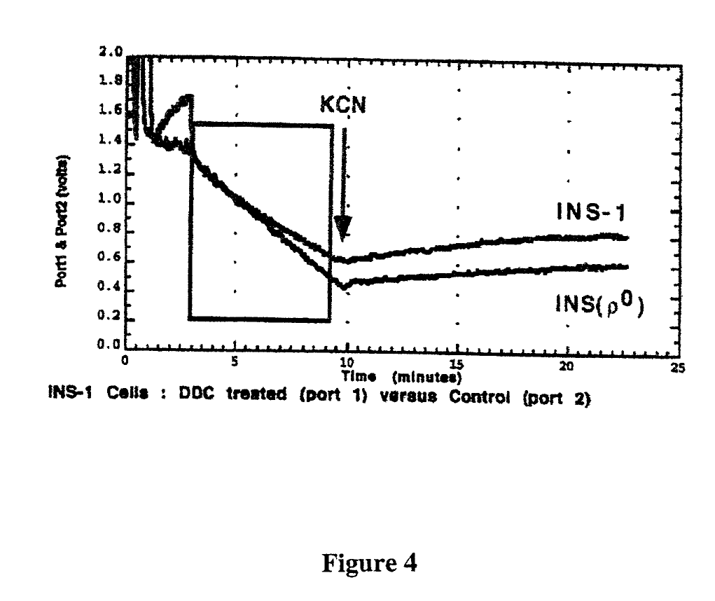

The results are shown in FIG. 4. In the boxed region of the plot, oxygen is seen to be disappearing from the media at a constant rate. After the addition of KCN, oxygen consumption rates plateau in a comparable manner in the two cell collections, indicating that mitochondrial-mediated respiration (Complex IV activity) was present in both the parent (untreated) INS-1 cells and in the ddC-treated cells (Rho revertants).

TABLE 3
LACTATE PRODUCTION IN DDC-TREATED CELLS OVER TIME

| | | | | |
|---|---|---|---|---|
| | | [glucose] | [lactate] | [lactate] |
| Time | Cells | mM | (mg/ml) | (mg/cell) |

| | | | | | |
|---|---|---|---|---|---|
| 40 | days | Untreated | 10 | 9.1 | 3.4 |
| | | ddC-treated | 10 | 2.8 | 2.8 |
| | | Untreated | 25 | 9.0 | 3.5 |
| | | ddC-treated | 25 | 25.6 | 18.6 |
| | | Untreated | 50 | 14.2 | 5.9 |
| | | ddC-treated | 50 | 20.4 | 27.2 |
| >50 | days | Untreated | 10 | 6.7 | 2.7 |
| | | ddC-treated | 10 | 4.2 | 2.0 |
| | | Untreated | 25 | 18.5 | 5.5 |
| | | ddC-treated | 25 | 20.1 | 7.7 |
| | | Untreated | 50 | 31.7 | 14.7 |
| | | ddC-treated | 50 | 20.2 | 9.3 |

From the foregoing it will be appreciated that, although specific embodiments of the invention have been described herein for purposes of illustration, various modifications may be made without deviating from the spirit and scope of the invention. Accordingly, the invention is not limited except as by the appended claims.

We claim:

1. A method of generating a ρ⁰ cell comprising:
contacting an insulin secreting cell with an antiviral compound.

2. A method of generating a mitochondrial DNA depleted cell comprising:
contacting an insulin secreting cell with an antiviral compound.

3. The method of either claim 1 or claim 2 wherein the antiviral compound is a nucleoside, nucleotide or base analog, or a prodrug thereof.

4. The method of claim 3 wherein the antiviral compound is selected from the group consisting of 2′,3′-dideoxycytidine (ddC), 3′-azido-3′ deoxythymidine (AZT), 2′,3′-dideoxyadenosine (ddA), 2′,3′-dideoxyguanosine (ddG), 2′,3′-dideoxythymidine (ddT), 2′3′-deoxyinosine (ddI), 2′3′-didehydro-3′-deoxythimidine (d4T), 2′,3′-dideoxydidehydrothymidine, 2′,3′-dideoxydidehydrocytidine, ganciclovir, acycloguanosine, fialuridine (FIAU), -2′,3′-dideoxy-3′-thiacytidine (3TC), lobucavir, cidofovir (HPMPC), PMPA, abacivir (1 592U89), bis-POM PMEA (adefovir dipivoxil), gemcitabine and combinations thereof.

5. The method of either claim 1 or claim 2 wherein the insulin secreting cell is an immortalized cell line.

6. The method of either claim 1 or claim 2 wherein the insulin secreting cell is capable of being induced to differentiate.

7. The method of either claim 1 or claim 2 wherein the insulin secreting cell is undifferentiated.

8. A method of producing a cybrid cell line, comprising the steps of:
treating an insulin secreting cell line with an antiviral compound to convert said cell line into a ρ⁰ cell line; and
repopulating said ρ⁰ cell line with isolated mitochondria to form said cybrid cell line.

9. The method of claim 8 wherein the cybrid cell line has extramitochondrial genomic DNA and mitochondrial DNA of differing biological origins.

10. The method of claim 9 wherein the cybrid cell line has extramitochondrial genomic DNA from a first species and mitochondrial DNA from a second species.

11. The method of claim 10 wherein said first species is selected from the group consisting of mouse and rat, and said second species is selected from the group consisting of mouse, rat, rabbit, hamster, guinea pig and gerbil.

12. The method of claim 11 wherein said second species is rat.

13. The method of claim 12 wherein said rat is a BHE/cdb rat.

14. A method of producing a cybrid cell line, comprising the steps of:
treating an insulin secreting cell line with an antiviral compound to convert said cell line into a mitochondrial DNA depleted cell line; and
repopulating said mitochondrial DNA depleted cell line with isolated mitochondria to form said cybrid cell line.

15. The method of claim 14 wherein the cybrid cell line has extramitochondrial genomic DNA and mitochondrial DNA of differing biological origins.

16. The method of claim 15 wherein the cybrid cell line has extramitochondrial genomic DNA and mitochondrial DNA from xenogeneic species.

17. The method of claim 16 wherein the cybrid cell line has mitochondrial DNA from a rodent species.

18. The method of claim 17 wherein the cybrid cell line has mitochondrial DNA from a species selected from the group consisting of mouse, rat, rabbit, hamster, guinea pig and gerbil.

19. The method of claim 18 wherein the cybrid cell line has mitochondrial DNA from a BHE/cdb rat.

20. The method of either claim 8 or claim 14 wherein the antiviral compound is a nucleoside analog.

21. The method of claim 20 wherein the antiviral compound is selected from the group consisting of 2′,3′-dideoxycytidine (ddC), 3′-azido-3′ deoxythymidine (AZT), 2′,3′-dideoxyadenosine (ddA), 2′,3′-dideoxyguanosine (ddG), 2′,3′-dideoxythymidine (ddT), 2′3′-deoxyinosine (ddI), 2′3′-didehydro-3′-deoxythimidine (d4T), 2′,3′-dideoxydidehydrothymidine, 2′,3′-dideoxydidehydrocytidine, ganciclovir, acycloguanosine, fialuridine (FIAU), -2′,3′-dideoxy-3′-thiacytidine (3TC), lobucavir, cidofovir (HPMPC), PMPA, abacivir (1592U89), bis-POM PMEA (adefovir dipivoxil), gemcitabine and combinations thereof.

22. The method of either claim 8 or claim 14 wherein the insulin secreting cell line to be treated with an antiviral compound is an immortalized cell line.

23. The method of any one of claims 8-19, wherein the cybrid cell line is capable of secreting insulin.

24. The method of any one of claims 8-19 wherein the cybrid cell line is capable of responding to insulin.

25. The method of any one of claims 8-19 wherein the cell line is derived from a pancreatic beta cell.

26. The method of any one of claims 8-19 wherein said cell line is an undifferentiated cell line that is capable of being induced to differentiate.

27. The method of any one of claims 8-19 wherein said isolated mitochondria are obtained from a subject known to be afflicted with a disorder associated with a mitochondrial defect.

28. The method of any one of claims 9-13 or 15-19, wherein said extramitochondrial genomic DNA has its origin in an immortal cell line, and said mitochondrial DNA has its origin in a human tissue sample.

29. The method of claim 28 wherein said human tissue sample is derived from a patient having a disease that is associated with a mitochondrial defect.

30. A method of constructing an immortal cybrid cell line, comprising the steps of:
a) treating an immortal insulin secreting cell line with an antiviral compound to convert said cell line into an immortal ρ⁰ cell line; and
b) repopulating said immortal ρ⁰ cell line with mitochondria isolated from tissue of a patient afflicted with a disorder selected from the group consisting of diabetes mellitus, Alzheimer's Disease, Parkinson's Disease, Huntington's disease, dystonia, Leber's hereditary optic neuropathy, schizophrenia, myoclonic epilepsy lactic acidosis and stroke (MELAS), and myoclonic epilepsy ragged red fiber syndrome (MERRF), NARP (Neuropathy; Ataxia; Retinitis Pigmentosa), MNGIE (Myopathy and external ophthalmoplegia; Neuropathy; Gastro-Intestinal; Encephalopathy), Kearns-Sayre disease, Pearson's Syndrome, PEO (Progressive External Ophthalmoplegia); congenital muscular dystrophy with mitochondrial structural abnormalities, Wolfram syndrome (DIDMOAD, Diabetes Insipidus, Diabetes Mellitus, Optic Atrophy, Deafness), Leigh's Syndrome, fatal infantile myopathy with severe mtDNA depletion, benign “later-onset” myopathy with moderate reduction in mtDNA, dystonia, arthritis, and mitochondrial diabetes and deafness (MIDD), to form said cybrid cell line.

31. A method of constructing an immortal cybrid cell line, comprising the steps of:
a) treating an immortal insulin secreting cell line with an antiviral compound to convert said cell line into an immortal mitochondrial DNA depleted cell line; and
b) repopulating said immortal mitochondrial DNA depleted cell line with mitochondria isolated from tissue of a patient afflicted with a disorder selected from the group consisting of diabetes mellitus, Alzheimer's Disease, Parkinson's Disease, Huntington's disease, dystonia, Leber's hereditary optic neuropathy, schizophrenia, myoclonic epilepsy lactic acidosis and stroke (MELAS), and myoclonic epilepsy ragged red fiber syndrome (MERRF), NARP (Neuropathy; Ataxia; Retinitis Pigmentosa), MNGIE (Myopathy and external ophthalmoplegia; Neuropathy; Gastro-Intestinal; Encephalopathy), Kearns-Sayre disease, Pearson's Syndrome, PEO (Progressive External Ophthalmoplegia); congenital muscular dystrophy with mitochondrial structural abnormalities, Wolfram syndrome (DIDMOAD, Diabetes Insipidus, Diabetes Mellitus, Optic Atrophy, Deafness), Leigh's Syndrome, fatal infantile myopathy with severe mtDNA depletion, benign “later-onset” myopathy with moderate reduction in mtDNA, dystonia, arthritis, and mitochondrial diabetes and deafness (MIDD), to form said cybrid cell line.

32. A method of preparing a cybrid animal, comprising the steps of:
a) treating embryonic cells isolated from a multicellular, non-human animal with an antiviral compound, thus converting said cells to a ρ⁰ state; and
b) repopulating said ρ⁰ embryonic cells with mitochondria isolated from another cell source, to produce said cybrid animal.

33. A method of preparing a cybrid animal, comprising the steps of:
a) treating embryonic cells isolated from a multicellular, non-human animal with an antiviral compound, thus converting said cells to a mitochondrial DNA depleted state; and
b) repopulating said mitochondrial DNA depleted embryonic cells with mitochondria isolated from another cell source, to produce said cybrid animal.

34. A method of detecting a disease associated with altered mitochondrial function comprising:
treating an insulin secreting cell line with an antiviral compound to convert said cell line into a mitochondrial DNA depleted cell line or a ρ⁰ cell line;
repopulating said mitochondrial DNA depleted cell line or ρ⁰ cell line with mitochondria from a donor subject suspected of having a disease associated with altered mitochondrial function to produce a cybrid cell line;
determining altered levels of insulin secretion by said cybrid cell line; and
therefrom identifying the mitochondria donor subject as having a disease associated with altered mitochondrial function.

35. A method of detecting a disease associated with altered mitochondrial function comprising:
treating an insulin secreting cell line with an antiviral compound to convert said cell line into a mitochondrial DNA depleted cell line or a ρ⁰ cell line;
repopulating said mitochondrial DNA depleted cell line or ρ⁰ cell line with mitochondria from a donor subject suspected of having a disease associated with altered mitochondrial function to produce a cybrid cell line;
comparing altered levels of insulin secretion by said cybrid cell line to insulin secretion by an insulin secreting cell line having mitochondria from a subject with normal mitochondrial function; and
therefrom identifying the mitochondria donor subject as having a disease associated with altered mitochondrial function.

36. A method of evaluating an antiviral compound for its effect on mitochondrial function, comprising:
treating an insulin secreting cell line with an antiviral compound to convert said insulin secreting cell line into a mitochondrial DNA depleted cell line or a ρ⁰ cell line;
repopulating said mitochondrial DNA depleted cell line or ρ⁰ cell line with mitochondria to produce a cybrid cell line; and
determining insulin secretion by said cybrid cell line in the presence or absence of an antiviral compound, therefrom identifying an effect of said antiviral compound on mitochondrial function.

37. The method of claim 36 wherein said mitochondria are from a subject suspected of having a disease associated with altered mitochondrial function.

38. The method of claim 36 wherein said cybrid cell line has extramitochondrial genomic DNA and mitochondrial DNA of differing biological origins.

39. The method of claim 38 wherein the cybrid cell line has extramitochondrial genomic DNA from a first species and mitochondrial DNA from a second species.

40. The method of claim 39 wherein said first species is selected from the group consisting of mouse and rat, and said second species is selected from the group consisting of mouse, rat, rabbit, hamster, guinea pig and gerbil.

41. The method of claim 40 wherein said second species is rat.

42. The method of claim 41 wherein said rat is a BHE/cdb rat.

43. A method of identifying an agent that at least partially restores insulin secretion to a cell exposed to an antiviral compound which inhibits insulin secretion, comprising:
treating an insulin secreting cell line with an antiviral compound to convert said cell line into a mitochondrial DNA depleted cell line or a ρ⁰ cell line;
repopulating said mitochondrial DNA depleted cell line or ρ⁰ cell line with mitochondria to produce a cybrid cell line;
contacting said cybrid cell line with a candidate agent capable of at least partially restoring insulin secretion to said cybrid cell line;
detecting an increase in insulin secretion by said cybrid cell line; and
therefrom identifying an agent that partially restores insulin secretion.

44. A method for selecting a therapeutic agent suitable for use in a subject having a disease associated with altered mitochondrial function, comprising:
treating an insulin secreting cell line with an antiviral compound to convert said cell line into a mitochondrial DNA depleted cell line or a ρ⁰ cell line;
repopulating said mitochondrial DNA depleted cell line or ρ⁰ cell line with mitochondria from a subject having a disease associated with altered mitochondrial function to produce a cybrid cell line;
detecting the level of insulin secretion by said cybrid cell line;
contacting said cybrid cell line with a candidate therapeutic agent;
detecting the effect of said candidate therapeutic agent on insulin secretion by said cybrid cell line; and
therefrom determining the suitability of the therapeutic agent.

45. A method for selecting a suitable therapeutic agent for use in a subject having a disease associated with impaired insulin secretion, comprising:
treating an insulin secreting cell line with an antiviral compound to convert said cell line into a mitochondrial DNA depleted cell line or a ρ⁰ cell line;
repopulating said mitochondrial DNA depleted cell line or ρ⁰ cell line with mitochondria from a subject having a disease associated with impaired insulin secretion to produce a cybrid cell line;
detecting the level of insulin secretion by said cybrid cell line;
contacting said cybrid cell line with a candidate therapeutic agent;
detecting the effect of said candidate therapeutic agent on insulin secretion by said cybrid cell line; and
therefrom determining the suitability of the therapeutic agent.

46. A method of evaluating the suitability of an antiviral compound for use in treating a virally infected patient, comprising determining the amount of mitochondrial DNA in at least one insulin secreting cell before and after contacting a candidate antiviral compound with said at least one insulin secreting cell, and therefrom determining the suitability of the antiviral compound for treating the patient.

47. A method of evaluating the suitability of an antiviral compound for use in treating a virally-infected patient, comprising determining the amounts of (i) mitochondrial DNA in, and (ii) insulin secreted by at least one insulin secreting cell before and after contacting a candidate antiviral compound with said cell, and therefrom determining the suitability of the antiviral compound for treating the patient.

48. The method of either claim 46 or claim 47 wherein said patient has a disease associated with impaired insulin secretion.

49. A method of evaluating a modification to an antiviral compound to determine if said modification alters side effects associated with said antiviral compound, comprising:
comparing a difference d for each of a first and second candidate agent, said candidate agent selected from the group consisting of the antiviral compound and a candidate antiviral compound comprising the modification, using the formula:
d=m2−m1
wherein
m1 is a ratio calculated using the formula:
m1=b/a
wherein
a is the amount of mitochondrial DNA in a first cell population comprising insulin secreting cells before contacting said cells with the candidate agent, and
b is the amount of mitochondrial DNA in said first cell population after contacting said cells with the candidate agent,
and wherein m2 is a ratio calculated using the formula:
m2=e/c
wherein
c is the amount of mitochondrial DNA in a second cell population comprising rho revertants of said insulin secreting cells before contacting said cells with the candidate agent, and
e is the amount of mitochondrial DNA in said second cell population after contacting said cells with the candidate agent;
and therefrom determining if the modification alters side effects associated with the antiviral compound.

50. The method of claim 48, further comprising:
comparing a difference p for each of a first and second candidate agent, said candidate agent selected from the group consisting of the antiviral compound and a candidate antiviral compound comprising the modification, using the formula:
p=q2−q1
wherein
q1 is a ratio calculated using the formula:
q1=s/r
wherein
r is the amount of insulin secreted by a first cell population comprising insulin secreting cells before contacting said cells with the candidate agent, and
s is the amount of insulin secreted by said first cell population after contacting said cells with the candidate agent,
and wherein q2 is a ratio calculated using the formula:
q2=u/t
wherein
t is the amount of insulin secreted by a second cell population comprising rho revertants of said insulin secreting cells before contacting said cells with the candidate agent, and
u is the amount of insulin secreted by said second cell population after contacting said cells with the candidate agent;
and therefrom determining if the modification alters side effects associated with the antiviral compound.

51. The method of any one of claims 46 to 50 wherein the antiviral compound is a nucleoside analog.
